(19) United States
(12) Patent Application Publication
(10) Pub. No.: US 2017/0159107 A1
(43) Pub. Date: Jun. 8, 2017

(54) UNHEATED EXTRACTION OF GENOMIC DNA IN AN AUTOMATED LABORATORY SYSTEM

(71) Applicant: True Health IP LLC, Frisco, TX (US)

(72) Inventor: Ross Higgins, Richmond, VA (US)

(21) Appl. No.: 15/435,622

(22) Filed: Feb. 17, 2017

Related U.S. Application Data
(63) Continuation of application No. 14/210,884, filed on Mar. 14, 2014.
(60) Provisional application No. 61/782,590, filed on Mar. 14, 2013.

(51) Int. Cl. C12Q 1/68

(52) U.S. Cl. CPC C12Q 1/6806

(57) ABSTRACT

A method for analyzing genomic DNA includes introducing a plurality of samples comprising human cells into individual vessels in each of a plurality of multi-vessel well plates. At least a subset of the human cells in the plurality of samples is lysed without the use of heat. DNA in the at least a subset of lysed human cells is isolated with the use of a plurality of paramagnetic beads. The isolated DNA is analyzed to identify one or more single nucleotide polymorphisms (SNPs), wherein the lysing, isolating, and analyzing steps are performed substantially in parallel for each of the plurality of samples.

This application is a continuation of U.S. patent application Ser. No. 14/210,884 filed on Mar. 14, 2014, which claims the benefit of U.S. Provisional Patent Application No. 61/782,590, filed on Mar. 14, 2013, which is hereby incorporated by reference in its entirety.

### FIELD

This technology generally relates to extraction of genomic DNA and, more particularly, to improved methods and devices for extracting genomic DNA from a sample without using heat.

### BACKGROUND

A wide variety of automated chemical analyzers are known in the art and are continually being improved to increase throughput, reduce turnaround time, and decrease requisite sample volumes. These analyzers conduct assays using reagents to identify analytes in biological fluid samples such as whole blood, blood serum, plasma and the like. The assay reactions generate various signals that can be manipulated to determine the concentration of analyte in the sample such as disclosed in U.S. Pat. Nos. 7,101,715 and 5,985,672, which are incorporated herein by reference. Improvements in analyzer technology, however, may be hampered if sufficient corresponding advances are not made in pre-analytical sample preparation and handling operations like sorting, batch preparation, centrifugation of sample tubes to separate sample constituents, cap removal to facilitate fluid access, extraction of cellular material, and the like.

To address deficiencies in sample preparation and handling operations, commercial automated pre-analytical sample preparation systems, such as automated liquid handlers available from Hamilton Robotics, Inc., of Reno, Nev., in combination with additional instruments, have been developed to automatically transport sample in tubes to a number of pre-analytical sample processing stations that have been “linked together” such as described in U.S. Pat. Nos. 6,984,527 and 6,442,440, which are incorporated herein by reference. These liquid handlers process a number of different patient specimens contained in standard, bar code-labeled tubes. The bar code label contains an accession number coupled to demographic information that is entered into a central information system that tracks each sample along with test orders and other desired information. An operator places the labeled tubes onto the liquid handler system which automatically sorts and routes samples to the requisite processing devices for pre-analytical operations, such as decapping and aliquot preparation, prior to the sample being subjected to analysis by one or more analytical stations also “linked” to the liquid handling system. The possible aliquot preparations include cell lysis, DNA extraction, and DNA purification to facilitate downstream analysis of DNA sequences, for example.

In many clinical assays, genomic DNA is required for the clinical analysis to identify a patient genotype. Particular genotypes may be more or less susceptible to disease states. For example in single nucleotide polymorphism (SNP) genotyping, the genetic variation of a single base pair mutation is detected at a specific locus, usually consisting of two alleles. SNPs are known to be involved in the cause of many human disease states, or increased risk of certain diseases states. Furthermore, SNPs are of interest in the field of pharmacogenomics, where genetic differences in metabolic pathways can affect an individual's responses to drugs in terms of therapeutic effect and risk of adverse effects.

For example, Apoliprotein E (ApoE) is the primary apolipoproteins found in very low-density lipoproteain (VLDL) particles and chylomicrons, as well as VLDL remnant lipoproteins and high-density lipoproteins (HDL). It is not present on LDL particles. Nevertheless, it is the primary binding protein for LDL receptors in the liver, whereby it mediates lipid metabolism. A polymorphic gene (alleles ε2, ε3, and ε4) codes for 3 protein isoforms (E2, E3, and E4) and a patient's genotype (alleles) can be determined by gene amplification techniques. Since the genotype modulates a patient's atherogenic potential, the ApoE test can provide information regarding one's risk of developing coronary artery disease. Testing for ApoE also provides physicians with useful information when prescribing lipid-lowering drugs that are influenced by the ApoE genotype.

ApoE is a glycoprotein found (often in multiple copies) and the different isoforms alter plasma lipoprotein concentrations because they have different affinities for various membrane receptors and lipases. This phenotypic expression of the different isoforms varies according to diverse environmental stimuli or genetic associations. ApoE has two primary metabolic roles involving its receptor-binding and lipid-binding functions: (1) transport of neutral lipids from their site of synthesis, or absorption, to the tissues where lipids are stored, metabolized or excreted, and (2) delipidation and transport of neutral lipids, in particular cholesterol, from the peripheral organs to the liver for excretion. ApoE also modulates the activity of enzymes involved in lipid and lipoprotein metabolism, such as hepatic lipase (HL), lipoprotein lipase (LPL), cholesterol ester transfer protein (CETP) and lecithin: cholesterol acyltransferase (LCAT).

The three isoforms vary in the amino acids present at position 112 and 158 of the protein, leading to three homozygous (E4/E4, E3/E3, and E2/E2) and three heterozygous (E4/E3, E4/E2, and E3/E2) genotypes and phenotypes, resulting from simple co-dominant Mendelian inheritance of the gene. The ApoE genotypes include ApoE2 (E2/E2, E2/E3), ApoE3 (E3/E3, E2/E4), and ApoE4 (E3/E4, E4/E4).

Analysis of the ApoE genotype is clinically valuable for the assessment and treatment of patients at risk of cardiovascular disease. Assays to determine the ApoE genotype are run on many thousands of patient samples at a central diagnostic laboratory. To maintain a viable profit margin on the assay, given a declining reimbursement from federal health programs and health insurance, the efficiency of the assay is continually optimized. Assays are typically run in high volume, with minimal steps, transformations, personnel involvement and equipment. Steps in the pre-analytical preparation of the sample can also be optimized to reduce energy and equipment requirements.

A variety of SNP tests, similar to ApoE genotyping are clinically valuable and processed by central clinical laboratories at high volume and low cost. Some examples include genotyping for Factor V, Prothombrin, CYP2C19, and MTHFR expression and Warfarin sensitivity. Clinical tests are designed for rapid assays and accuracy. As part of the pre-analytical process, each test typically involves the extraction of genomic DNA.

Extraction of genomic DNA can be a cumbersome process which involves the lysis of cells, opening of the cell nucleus, and separation of the genomic DNA from all non-DNA particles. Many methods are known in the art for the performance of DNA extraction such as those disclosed in U.S. Pat. No. 6,423,488 and U.S. Patent Application Publication No. 2004/0265855, which are incorporated herein by reference.

While high throughput methods for sequence detection are available, no comparable methods exist for the extraction of DNA useful in a high throughput assay for sequence detection. Rather, existing DNA extraction methods are still labor intensive and time consuming. Many extraction methods require the DNA samples to be treated in individual tubes. Samples are subjected to a number of steps, including proteinase digestion, extraction with organic solvents, and precipitation. The extraction step is particularly problematic because of the awkwardness of manipulation of the solution phases. Salting out has been used as an alternative for extraction of unwanted proteins, but this method involves multiple centrifugations and tube transfers. Kits are available which avoid the extraction steps by using DNA binding resins and allow for the processing of 96 samples at a time. However, the resins are not reusable, and their use can result in poor yield and inconsistent DNA quality. In addition, these kits are not cost-effective, costing up to $3.00 per sample processed for extraction.

A protocol for alkaline lysis has, for instance, been described in Sambrook et al., “Molecular Cloning, A Laboratory Handbook”, CSH Press, Cold Spring Harbor 1989 or Ausubel et al., “Current Protocols, in Molecular Biology”, John Wiley & Sons, Inc., N.Y. 2002. Methods for purifying DNA, RNA, or their hybrids with magnetic silica beads have been described for example in U.S. Pat. No. 6,027,945 and International Patent Application No. PCT/US98/01149 entitled “Methods of Isolating Biological Target Materials Using Silica Magnetic Particles” and published as Publication No. WO 98/31840, which are incorporated herein by reference. Removing cell debris by using magnetic micro-particles has been disclosed in U.S. Pat. No. 5,646,283, which is incorporated herein by reference.

Prior high-throughput methods used in central laboratories include the application of proteinase K to facilitate the lysis of cells and destruction of cell debris in the process of isolating genomic DNA. Proteinase K is a broad-spectrum serine proteinase used in molecular biology to digest protein and remove contamination from preparations of nucleic acid. Addition of Proteinase K to nucleic acid preparations inactivates nucleases that might otherwise degrade the DNA or RNA during purification. Proteinase K is suited to this application since the enzyme is active in the presence of chemicals that denature proteins, such as SDS and urea, chelating agents such as EDTA, sulfhydryl reagents, as well as trypsin or chymotrypsin inhibitors. Proteinase K is used for the destruction of proteins in cell lysates (tissue, cell culture cells) and for the release of nucleic acids, since it very effectively inactivates DNases and RNases. Proteinase K is very useful in the isolation of highly native, undamaged DNAs or RNAs, since most microbial or mammalian DNases and RNases are rapidly inactivated by the enzyme, particularly in the presence of 0.5-1% SDS. Genomic DNA can be purified from a saturated liquid culture by being lysed where proteins are removed by a digest with 100 μg/ml Proteinase K for 1 h at 37° C. However, the heating step in the use of proteinase K requires additional equipment and energy inputs in the process of DNA isolation, which is undesirable.

Most methods incorporate a heating step to facilitate the breakdown of cell membranes and digestion of contaminant proteins. A wide survey of protocols that include a detergent such as SDS to aid in cell lysis and a proteinase such as proteinase K in the literature indicates that the use of heat in extraction is a universal component of extraction protocols. However, heat in a high-throughput system substantially increases complexity and cost-of-use. It is believed that no entity to date has proposed an unheated high-throughput DNA extraction system and method, which may process, for example 100, 200, 300, 400, 500, 1000, 2000, 3000, 4000 or more samples in a 24-hour period.

An automated high through-put DNA preparation system for the use of microtiter plates has been disclosed in European Patent Application Publication No. 569,115. By integrating modified centrifuges, a DNA preparation after alkaline lysis is made possible. However, a high degree of purity of the DNA, desired for optimal DNA amplification, is not achieved due, at least in part, to the fact that the DNA is still contaminated by endotoxins. It is also disadvantageous that this system, along with the Genesis™ system available from Tecan Inc. of Switzerland and the Biomek 2000™ system available from Beckman Coulter, Inc. of Brea, Calif., for example, are not outlined as conveyor road systems or can be enlarged as such. It is therefore not possible to interconnect the individual process steps using these systems.

Additionally, a variety of instruments and methods to perform DNA purification are known in the art. These include paramagnetic bead-based separation technologies such as the MagnaPure™ DNA purification kits available from F. Hoffmann-La Roche Ltd. of Switzerland, which have been used in the past for the extraction and purification of genomic DNA. However, these methods are not fully automated from start to finish and require many manual steps of pipetting, mixing and sample transferring without the reassurance of barcode reading, mapping and linking. Accordingly, while current automated DNA extraction technologies are low-to-medium throughput, due to the rapid growth and high throughput nature of central clinical diagnostic laboratories and needs, a faster method is needed. This invention answers that need.

### SUMMARY

This invention relates to a method for analyzing genomic DNA includes introducing a plurality of samples comprising human cells into individual vessels in each of a plurality of multi-vessel well plates. At least a subset of the human cells in the plurality of samples is lysed without the use of heat. DNA in the at least a subset of lysed human cells is isolated with the use of a plurality of paramagnetic beads. The isolated DNA is analyzed to identify one or more single nucleotide polymorphisms (SNPs), wherein the lysing, isolating, and analyzing steps are performed substantially in parallel for each of the plurality of samples.

In an aspect, this technology provides a rapid method for extracting and preparing DNA for use in a subsequent high-throughput genotyping assay. This technology is particularly useful for extracting DNA from human clinical samples of blood for use in a high throughput screening assay such as, for example, an assay to detect SNPs in the genome of a patient.

Additionally, this technology advantageously combines automated sample handling procedures including massively parallel pipetting, barcode scanning for tracking of samples, incubation/shaking steps, and magnetic purification. Accordingly, manual pipetting or manual matching of sample numbers is not required thereby increasing throughput and quality, particularly with respect to contamination and sample mix-ups. Additionally, the methods of this technology are advantageously performed at room temperature and without any heating. Accordingly, with this technology, extraction time can be reduced and samples can be processed in relatively less time.

### BRIEF DESCRIPTION OF THE DRAWINGS

FIG. 1 is a flowchart of an exemplary method for unheated extraction of genomic DNA;

FIG. 2 is an exemplary liquid handling system with an exemplary two-dimensional scanner;

FIG. 3 is an exemplary liquid handling system with exemplary magnetic devices;

FIG. 4 is an exemplary liquid handling system with exemplary source carriers; and

FIG. 5 is an exemplary liquid handling system with exemplary shaker devices.

### DETAILED DESCRIPTION

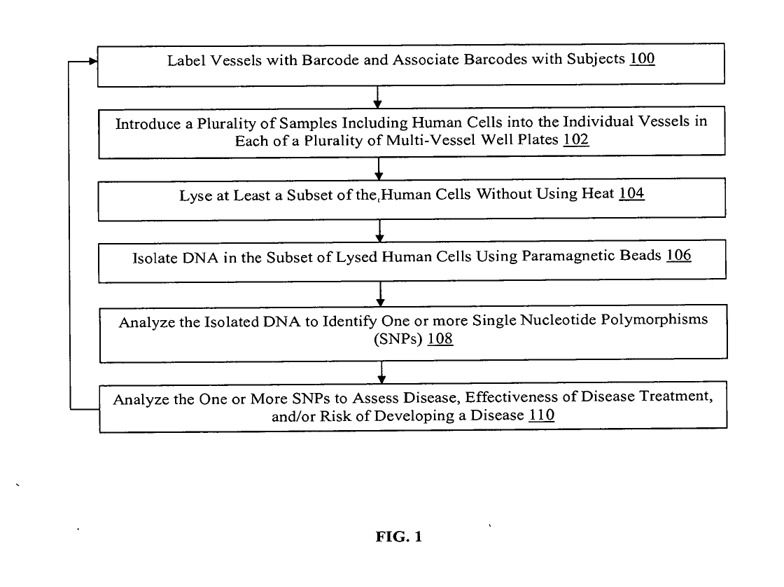

Referring to FIG. 1, a flowchart of an exemplary method for unheated extraction of genomic DNA is illustrated. The steps of the method described and illustrated below with reference to FIG. 1 are performed at room temperature, without the introduction of heat from an external source. Additionally, at least steps 102-106 of the method described and illustrated with reference to FIG. 1 are performed in a single liquid sample handling instrument, such as a MicroLab Star™ platform liquid handling system available from Hamilton Robotics, Inc. of Reno, Nev., although other liquid handling systems can also be used. The method of the present invention is particularly useful for providing rapid extraction of DNA from human clinical samples for use in a high throughput screening assay as, for example, an assay to detect SNPs in the genome of a patient.

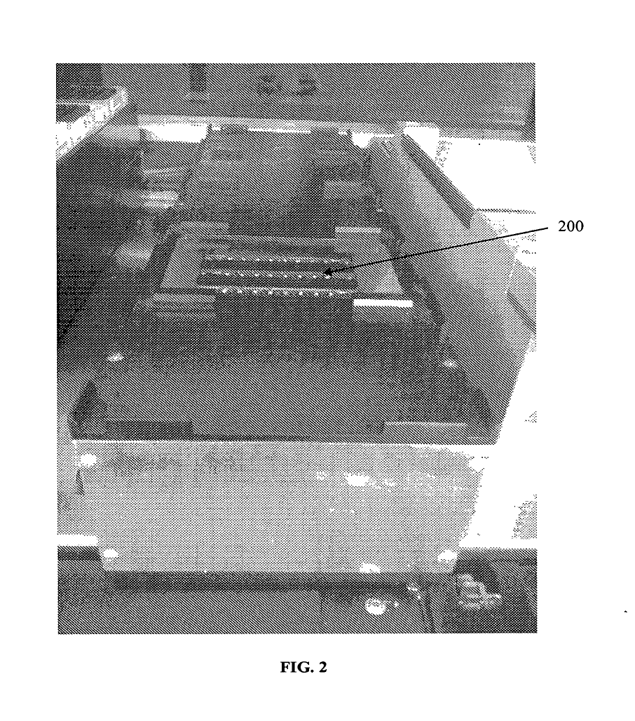

In step 100 in this example, one or more multi-vessel well plates, and/or associated vessels, are labeled with a bar code that is associated in a computing device with a unique patient or subject identifier. By labeling each of the multi-vessel well plates, the multi-vessel well plates can be tracked using the computing device as the multi-vessel well plates are processed. An exemplary two-dimensional scanner 200 of a liquid handling system is shown in FIG. 2. Optionally, four multi-vessel well plates are used for each iteration of the steps described and illustrated with reference to FIG. 1. In this example, the well plates are 96-sample multi-vessel well plates, although other numbers of well plates and other sizes of well plates can also be used.

In step 102 in this example, samples including human cells are introduced into the individual vessels in each of the plurality of labeled multi-vessel well plates. In this example, the samples including the human cells include body fluids, body wastes, body excretions, or blood, although other sample types can also be used.

In step 104 in this example, at least a subset of the human cells in the plurality of samples is lysed without the use of heat. In one example, the lysing includes introducing one or more chemical reagents to the plurality of samples. Exemplary chemical reagents can include a chaotropic salt solution, a protease enzyme, such as Proteinase K, or a combination thereof. Other chemical reagents and other protease enzymes can also be used.

In an unheated extraction step, a detergent solution is applied to the sample to effect cell lysis at room temperature. In some cases, the detergent concentration may be increased from that used in a heated method. Detergent and proteinase concentration may both be increased to complete unheated extraction. Generally, SDS (Sodium dodecyl sulfate) is used as an extraction detergent. A more aggressive detergent may be substituted into a lysis buffer or additional extraction reagents may be added, including deoxycholate, cholate, sarkosyl, triton X-100, DDM (n-Dodecyl β-D-maltoside), digitonin, tween 20, tween 80, CHAPS (3-[(3-cholamidopropyl)dimethylammonio]-1-propanesulfonate), and/or urea.

In some cases, the sample may be mixed with a first portion of detergent solution, agitated, mixed with a second portion of detergent solution and agitated again, such that repetition of detergent and agitation steps may replace a heating step. Repetitive aspiration by pipette can facilitate lysis instead of, or in addition to repetitive detergent additions. Subsequent to or at the same time as the detergent addition, proteinase K may be added to the sample solution to break down protein contaminants in solution. Typically, a heating step facilitates proteinase K activity through the denaturation of proteins in solution. In the high-throughput, automated system and method described here, however, the reagents may be applied at room temperature, or unheated, to conserve resources.

The removal of heating steps increases throughput by decreasing overall extraction time from at least 2.5 hours per incubation to less than 2 hours per incubation. The complete unheated extraction facilitates DNA extraction from a plurality of samples in a plurality of vessels in less than 2 hours. The samples may be in a 96-sample well container, with a plurality of 96-well containers per extraction run on the automated liquid handling system. In an 8-hour shift, at least one additional extraction run may be completed using the unheated method versus the standard heated method with a potential for 384 additional samples extracted in a sample handling unit handling 4 96-well plates. In a 24-hour period, 2-3 additional extraction runs may be completed, with a potential of >1000 additional samples extracted by each sample handling unit handling 4 96-well plates. The unheated high-throughput DNA extraction system and method may therefore facilitate extraction of 1000, 2000, 3000, 4000 or more samples on parallel liquid handling systems per 24-hour period.

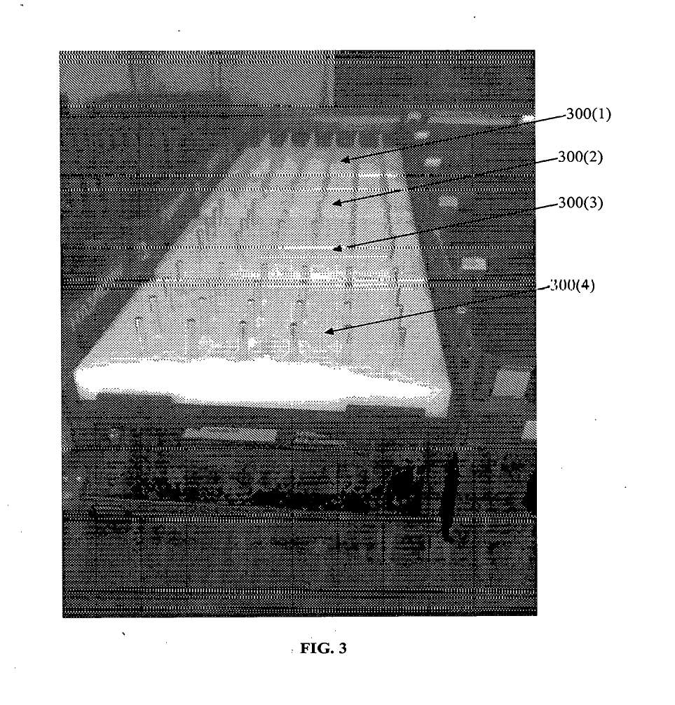

In step 106 in this example, DNA in the at least a subset of lysed human cells is isolated with the use of a plurality of paramagnetic beads. The paramagnetic beads can be Mag-Bind™ beads available from Omega Bio-Tek Inc. of Norcross, Ga., although other paramagnetic beads can also be used. The paramagnetic beads can be introduced to the multi-vessel well plates and attracted to four magnetic devices on each carrier of the liquid handling system. Exemplary magnetic devices 300(1)-(4) of a liquid handling system are shown in FIG. 3. Accordingly, the liquid handling system can include a magnetic device 300(1)-(4) for each of the four multi-vessel well plates used in this example. Each of the magnetic devices 300(1)-X(4) includes 24 magnetic vertical prongs and, accordingly, each prong fits between four wells on the 96-sample multi-vessel well plates.

The paramagnetic beads of the vertical prongs in this example are static, although in other examples other orientations and/or other mobile magnets or paramagnetic particles could also be used. The paramagnetic beads in this example allow for rapid isolation of high quality genomic DNA from 1-200 μL of whole blood samples utilizing reversible binding properties. The isolated DNA can be used without modifications in downstream applications such as Polymerase Chain Reaction (PCR), for example.

Optionally, unbound substances such as proteins, polysaccharides, and cellular debris, for example, are removed by a high salt wash and/or an ethanol wash, for example, although other methods for washing unbound substances can also be used. The isolated DNA can then be eluted from the paramagnetic beads in a low ionic strength buffer, for example, although other elution methods can also be used.

In step 108, the isolated DNA is analyzed to identify one or more single nucleotide polymorphisms (SNPs). The SNPs can include APOE 112, APOE 158, MTHFR C677T, FII, FVL, CYP2C19*2, CYP2C19*3, CYP2C19*17, CYP2C9*2, CYP2C9*3, and/or VKORC1, for example, although other SNPs can also be identified from the isolated DNA.

In step 110, the one or more SNPs identified in step 108 are analyzed to assess disease state, effectiveness of disease treatment, and/or risk of developing a disease, for example. Exemplary diseases can include cardiovascular disease, diabetes, or fatty liver disease, for example, although the SNPs can also be used to asses other diseases.

### EXAMPLE 1

In one exemplary implementation of steps 102-106 of FIG. 1, the supplies, equipment, and reagents included in Table 1 are used, although other supplies, equipment, and/or reagents from other vendors could also be used.

TABLE 1
Supplies
CO-RE Tips 12 × 480 Standard Volume (300 μL) with Filter
CO-RE Tips 8 × 480 Standard Volume (1000 μL) with Filter
Reagent container (50 mL)
Waste bags
Cap Holder Racks 2 × 10 RNAse/DNAse/Pyrogen-free
Matrix 0.5 mL 2D Screw tubes PP, V Bottom with Cap-Latch Rack
Plate, 96 Deep Well, 1.2 mL
Axygen Reservoir 96 Row, Pyramid Bottom, Single Well, Sterile
Thermo Clear Seal 3730
BD Sterile Culture Tubes 12 × 75
Adhesive Covers (similar alternative is suitable)
Equipment list
MicroLab Star/StarLet Liquid Handling System (Hamilton Robotics,
Inc.)
ALPS-3000 Heat Sealer (Thermo Fisher Scientific Inc.)
Compact 106 Air Compressor
InfinityXL Platform Rocker (Next Advance, Inc.)
Nexar (Douglas Scientific)
Capper/Decapper unit (Hamilton Robotics, Inc.)
Reagents
Mag-Bind Blood DNA HDQ Kit and Proteinase K (Omega Bio-Tek
Inc.)
Ethanol (Anhydrous Alcohol) C2H5OH (IBI Scientific)
Isopropyl Alcohol (Isopropanol) C3H7OH (IBI Scientific)
Molecular grade (nuclease free) glass distilled reagent water
(Teknova)

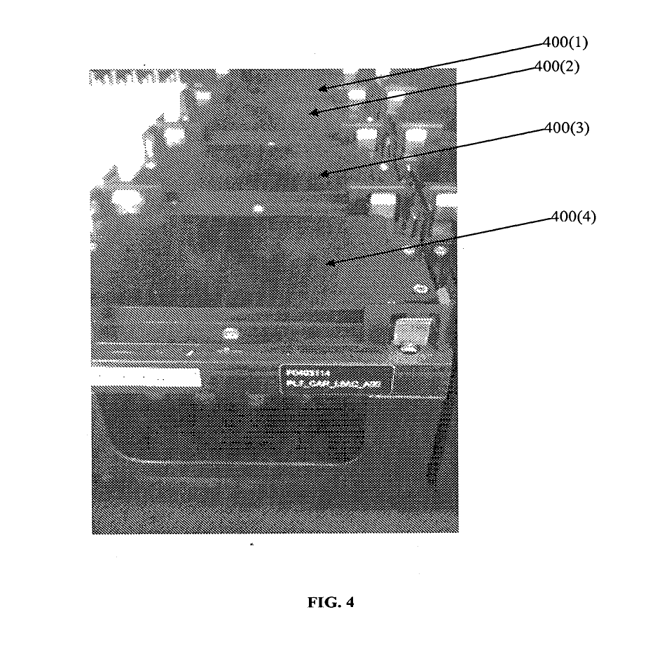

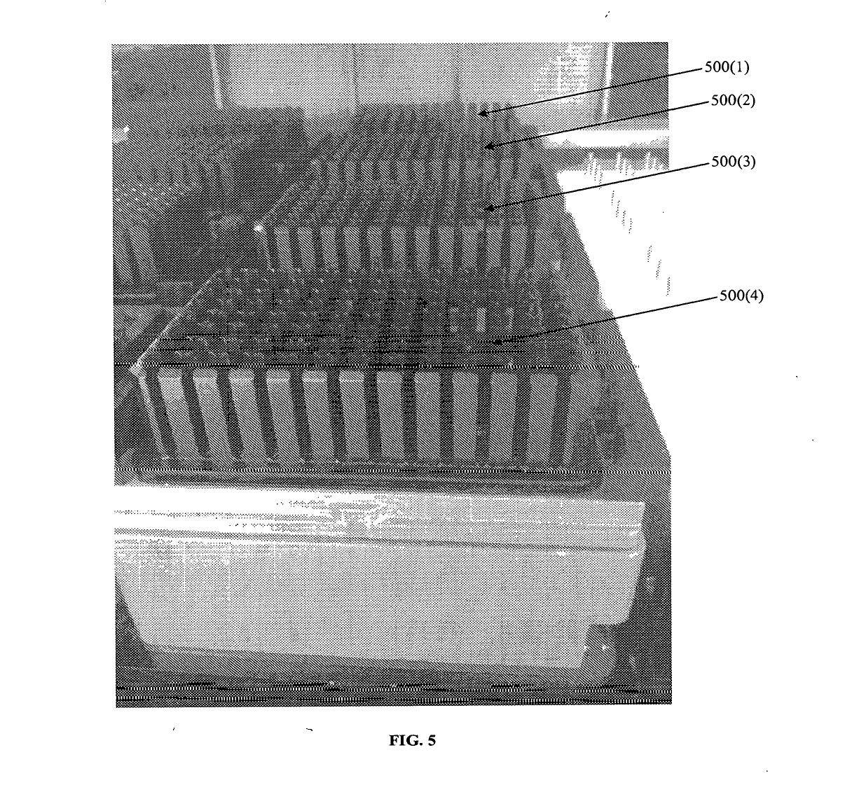

In this example, in step 102, 96-sample multi-vessel well plates with blood are loaded into a source carrier of a liquid handling system. Exemplary source carriers 400(1)-400(4) of a liquid handling system are shown in FIG. 4. Next, the well plates are transported to a shaking device, shaken, and transported back to the source carrier. Exemplary shaker devices 500(1)-500(4) of a liquid handling system are shown in FIG. 5.

In step 104 in this example, a lysis buffer containing a chaotropic salt, such as guanidinium hydrocholoride, is added to a reagent reservoir of the liquid handling system, which then aspirates the reagent and dispenses into the well plates. The well plates are then transported by the liquid handling system to shakers, shaken, and transported back to the source carrier.

In step 106 in this example, a Mag-Bind™ HDQ mix (prepared as a mastermix with HDQ Beads, Isopropanol and HDQ binding buffer) is added to a reagent reservoir of the liquid handling system. The liquid handling system then aspirates the HDQ Mix and dispenses into the well plates. Next, the well plates are transported to shakers, shaken, and transported to magnets for magnetic separation, and then the liquid handling system then aspirates waste from the well plates.

In this example, aqueous Guanidine Hydrochloride solution (VHB) buffer is then added to the reagent reservoir of the liquid handling system which then aspirates and dispenses into the well plates. Subsequently, the well plates are transported to shakers, shaken, and transported to the magnets for magnetic separation, and then the liquid handling system aspirates waste from the well plates. Optionally, more VHB buffer can be added and the aspirating, dispensing, transporting to the shaker, shaking, and transporting to the magnets, and aspirating waste steps can be repeated one or more times.

Subsequent to utilizing the VHB buffer, in this example an SPM wash buffer is added to the reagents reservoir of the liquid handling system which then aspirates, and dispenses into the well plates. Subsequently, the well plates are transported to shakers, shaken, and transported to the magnets for magnetic separation, and then the liquid handling system then aspirates waste from the well plates.

Finally, in this example, an elution buffer can be added to the reagent reservoir of the liquid handling system which then aspirates and dispenses into the well plates. Subsequently, the well plates are transported to shakers, shaken, and transported to the magnets for magnetic separation, and then the liquid handling system aspirates waste from the well plates. Accordingly, any number of buffers can be used in the isolation of the DNA. Additionally, the shakers are not heated in this example. With this technology, at least 4000 samples can advantageously be analyzed in a 24 hour period.

In order to analyze the efficacy of this example, genomic DNA from whole blood samples was isolated using the methods described and illustrated in this example and a reference method, using the same liquid handling system, analyzed for the APOE 112, APOE 158, MTHFR C677T, FII, FVL,CYP2C19*2, *3 and *17, and Warfarin (CYP2C9*2, *3 and VKORC1) SNPs, and the concordance was compared. The reference method included automated transfers of sample materials wherein the materials are heated in either a water bath or on a heating block after addition of the lysis buffer.

At least 95% of the samples extracted using the method of this example (referred to herein as HDQ method) resulted in a genotype call for each one of the above-identified SNPs. There was no negative concordance in genotype results for all of the SNP assays between the two instruments. Samples that were marked as non-concordance/unable to assay had undetermined status for one of their results. The result of the comparison is illustrated in the following Tables 2-10.

TABLE 2

| APO-E 112 Summary | HDQ-Bahamas | HDQ-Haiti |
|---|---|---|
| Samples | 384 | 384 |
| Not Analyzed | 1 | 2 |
| n | 383 | 382 |
| Positive Concordance | 381 | 381 |
| Non Concordance | 2 | 1 |
| Negative Concordance | 0 | 0 |
| % Positive | 99.48% | 99.74% |
| Concordance | | |

TABLE 3

| APO-E 158 Summary | HDQ-Bahamas | HDQ-Haiti |
|---|---|---|
| Samples | 384 | 384 |
| Not Analyzed | 1 | 2 |
| n | 383 | 382 |
| Positive Concordance | 383 | 382 |
| Non Concordance | 0 | 0 |
| Negative Concordance | 0 | 0 |
| % Positive | 100.00% | 100.00% |
| Concordance | | |

TABLE 4

| CYP2C19*2 | | |
|---|---|---|
| Summary | HDQ-Bahamas | HDQ-Haiti |
| Samples | 380 | 380 |
| Not Analyzed | 3 | 4 |
| n | 377 | 376 |
| Positive Concordance | 376 | 376 |
| Non Concordance | 1 | 0 |
| Negative Concordance | 0 | 0 |
| % Positive | 99.73% | 100.00% |
| Concordance | | |

TABLE 5

| CYP2C19*3 | | |
|---|---|---|
| Summary | HDQ-Bahamas | HDQ-Haiti |
| Samples | 380 | 380 |
| Not Analyzed | 1 | 2 |
| n | 379 | 378 |
| Positive Concordance | 379 | 378 |
| Non Concordance | 0 | 0 |
| Negative Concordance | 0 | 0 |
| % Positive | 100.00% | 100.00% |
| Concordance | | |

TABLE 6

| CYP2C19*17 | | |
|---|---|---|
| Summary | HDQ-Bahamas | HDQ-Haiti |
| Samples | 380 | 380 |
| Not Analyzed | 2 | 3 |
| n | 378 | 377 |
| Positive Concordance | 376 | 376 |
| Non Concordance | 2 | 1 |
| Negative Concordance | 0 | 0 |
| % Positive | 99.47% | 99.73% |
| Concordance | | |

TABLE 7

| FVL Summary | HDQ-Bahamas | HDQ-Haiti |
|---|---|---|
| Samples | 380 | 380 |
| Not Analyzed | 1 | 2 |
| n | 379 | 378 |
| Positive Concordance | 377 | 378 |
| Non Concordance | 2 | 0 |
| Negative Concordance | 0 | 0 |
| % Positive | 99.47% | 100.00% |
| Concordance | | |

TABLE 8

| Factor II Summary | HDQ-Bahamas | HDQ-Haiti |
|---|---|---|
| Samples | 380 | 380 |
| Not Analyzed | 1 | 2 |
| n | 379 | 378 |
| Positive Concordance | 379 | 376 |
| Non Concordance | 0 | 2 |
| Negative Concordance | 0 | 0 |
| % Positive | 100.00% | 99.47% |
| Concordance | | |

TABLE 9

| MTHFR Summary | HDQ-Bahamas | HDQ-Haiti |
|---|---|---|
| Samples | 382 | 382 |
| Not Analyzed | 2 | 3 |
| n | 380 | 379 |
| Positive Concordance | 379 | 379 |
| Non Concordance | 1 | 0 |
| Negative Concordance | 0 | 0 |
| % Positive | 99.74% | 100.00% |
| Concordance | | |

TABLE 10

| Warfarin Summary | HDQ-Bahamas | HDQ-Haiti |
|---|---|---|
| Samples (all 3 SNPs) | 279 | 279 |
| Not Analyzed | 0 | 0 |
| n | 279 | 279 |
| Positive Concordance | 279 | 279 |
| Non Concordance | 0 | 0 |
| Negative Concordance | 0 | 0 |
| % Positive | 100.00% | 100.00% |
| Concordance | | |

Accordingly, by this technology, DNA can be rapidly extracted from a human sample and prepared for use in a subsequent high-throughput genotyping assay is provided. With this technology, cells are lysed without requiring heat thereby reducing the time and required energy for performing the lysing. Additionally, all of the steps required to isolate the DNA can be performed on the same liquid handling system using a bar code tracking system thereby avoiding the need for manual pipetting and manual matching of sample numbers. Accordingly, extraction time can be significantly reduced and throughput can be increased, thereby allowing more samples to be analyzing over the same period of time.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for analyzing genomic DNA, the method comprising:
introducing a plurality of samples comprising human cells into individual vessels in each of a plurality of multi-vessel well plates;
lysing at least a subset of the human cells in the plurality of samples without the use of heat;
isolating DNA in the at least a subset of lysed human cells with the use of a plurality of paramagnetic beads; and
analyzing the isolated DNA to identify one or more single nucleotide polymorphisms (SNPs), wherein the lysing, isolating, and analyzing steps are performed substantially in parallel for each of the plurality of samples.

2. The method of claim 1, wherein the lysing further comprises introducing one or more chemical reagents to the plurality of samples.

3. The method of claim 2, wherein the one or more chemical reagents comprises a chaotropic salt solution.

4. The method of claim 2, wherein the one or more chemical reagents comprises a protease enzyme.

5. The method of claim 4, wherein the protease enzyme is Proteinase K.

6. The method of claim 1, further comprising labeling each of the plurality of multi-vessel well plates with a barcode.

7. The method of claim 6, further comprising scanning the barcodes and associating the barcodes with a unique patient sample identifier in a computing device.

8. The method of claim 7, further comprising tracking each of the plurality of multi-vessel well plates with the computing device as the plurality of multi-vessel well plates are processed by an automated liquid handling system.

9. The method of claim 1, further comprising performing the introducing, lysing, isolating, and analyzing steps for at least 4000 samples in a 24 hour period.

10. The method of claim 1, further comprising analyzing the one or more SNPs to assess cardiovascular health, effectiveness of a cardiovascular disease treatment, or a risk of developing cardiovascular disease for a subject.

11. The method of claim 1, further comprising analyzing the one or more SNPs to assess diabetes, effectiveness of a diabetes treatment, or a risk of developing diabetes for a subject.

12. The method of claim 1, further comprising analyzing the one or more SNPs to assess fatty liver health, effectiveness of a fatty liver disease treatment, or a risk of developing fatty liver disease for a subject.

13. The method of claim 1, wherein the one or more SNPs are selected from the group consisting of APOE 112, APOE 158, MTHFR C677T, FII, FVL, CYP2C19*2, CYP2C19*3, CYP2C19*17, CYP2C9*2, CYP2C9*3 and VKORC1.

14. The method of claim 1, wherein the plurality of samples are selected from the group consisting of body fluids, body wastes, body excretions, and blood.

15. The method of claim 1, wherein the plurality of samples comprise blood.

16. The method of claim 1, wherein the introducing, lysing, isolating, and analyzing steps arc performed at room temperature.

17. The method of claim 1, wherein one or more of the introducing, lysing, isolating, and analyzing steps are performed in a single liquid sample handling instrument.

18. The method of claim 1, wherein each of a plurality of multi-vessel well plates comprise 96-sample multi-vessel well plates.
